(19) United States
(12) Patent Application Publication
(10) Pub. No.: US 2017/0065233 A1
(43) Pub. Date: Mar. 9, 2017

(54) Image Guided Small Animal Stereotactic Radiation Treatment System

(71) Applicant: UNIVERSITY OF MIAMI, Miami, FL (US)

(72) Inventor: Yidong Yang, Miami, FL (US)

(21) Appl. No.: 15/255,735

(22) Filed: Sep. 2, 2016

Related U.S. Application Data
(60) Provisional application No. 62/214,003, filed on Sep. 3, 2015.

(51) Int. Cl. A61B 6/03; A61B 6/00; A61N 5/10; A61B 6/06

(52) U.S. Cl. CPC A61B 6/032; A61B 6/06; A61B 6/4085; A61N 5/10; A61B 6/5211; A61B 6/508

(57) ABSTRACT

A dual, imaging and radiation system provides for finely aligned movement of a subject through the use of a computer controlled mounting stage having separate, non-gantry translational and rotational controllable movement. The system cycles a subject tissue between a treatment position where radiation doses are applied and an imaging position where high-quality computed tomography (CT) imaging is performed. Selective dose profiles and dose depths are achievable around an isocenter of the system.

### CROSS-REFERENCE TO RELATED APPLICATION

This application claims the benefit of U.S. Provisional Application Ser. No. 62/214,003, filed Sep. 3, 2015, entitled “Image Guided Small Animal Stereotactic Radiation Treatment System,” which is hereby incorporated by reference in its entirety.

### FIELD OF THE DISCLOSURE

The present disclosure relates generally to techniques for stereotactic radiation therapy and, more particularly, to stereotactic radiation treatment systems providing both treatment operation and imaging operation.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

In radiotherapy, clinicians and researchers rely on translational radiation studies performed on laboratory animals to investigate radiobiology hypothesis, to discover new cancer biomarkers, and to develop novel therapeutic strategies. These translational studies should be performed on small animal irradiators that can deliver focal irradiation to the tumor target while sparing healthy tissues. Most radiation research labs, however, do not have access to irradiators with such capability.

Products with computed tomography (CT) image guidance have been developed to achieve better tumor targeting. But they are too expensive for most labs to afford and too complex for common researchers to operate. There is a need for cost effective techniques for targeted translational radiation apparatuses and studies that allow for accurate targeting, but that are not exorbitant in cost or complex in operation.

### SUMMARY OF THE INVENTION

To address these challenges, the application describes a medical imaging, guided small animal irradiator system capable of high quality imaging and precision radiation targeting. The system, which in an example implementation is referred herein as the iSMAART system, utilizes a unique imaging and irradiation geometry, that includes a stationary x-ray tube, a stationary flat panel detector, and a rotatable and translational animal stage.

The dual imaging and radiation systems described herein can dramatically reduce total cost translational studies and significantly improve the stability and robustness of the system. Our testing further shows that the system provides superior functionality in high-quality CT imaging and precision stereotactic radiation with both phantom and animal experiments, when compared to existing systems. In an example implementation, e.g., the targeting precision for CT guided focal irradiation was below 0.2 mm.

In accordance with an embodiment, an x-ray radiation treatment and analysis system for use in vivo testing of a subject tissue, the system comprises: a subject mounting stage having a rotational subsystem configured to rotate about a central axis and having a translational subsystem configured to move in translatory manner along at least two orthogonal directions; an image plate configured to image computed tomography images or a planar radiograph collected of a tissue of the subject during an imaging mode of the system; a collimating stage mounted to receive x-ray radiation from an x-ray source and configured to collimate the received x-ray radiation into a desired aperture size and direct the collimated x-ray radiation onto the tissue of the subject, where the collimating stage is adapted (i) to adjust between a first desired aperture size for use during an imaging mode and a second desired aperture size for use during a treatment mode, or (ii) to automatically move out of a path of the x-ray radiation during the imaging mode, wherein the image plate and the collimating stage are maintained in a fixed, not moving position during the imaging mode and during the treatment mode, and wherein the subject mounting stage is controlled to move to place the subject in proper position for imaging during the image mode and for treatment during the treatment mode; and a controller having at least one processor and at least one memory, the controller being coupled to control movement of the subject mounting stage in switching between operation of the system in the imaging mode and the treatment mode.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 illustrates a small-animal imaging and radiation treatment system, in accordance with an example.

FIG. 2A illustrates a transverse plane of a cone beam computed tomography (CBCT) technique of an image quality phantom. FIG. 2B illustrates the modulation transfer function (MTF) from a wire based oversampling, in accordance with an example.

FIGS. 3A-3C are images of three different prostate tumors excised for testing. FIGS. 3D-3F are 3D renders of tumor contour images contoured from CBCT collected images. FIGS. 3G-3I are three orthogonal CBCT slices of the first tumor, in FIG. 3A, with arrows indicating the tumor edge enhanced.

FIG. 4 illustrates exposure profiles for two different collimators for images taken from two different directions, i.e., AP represents images taken from a collimator in the anterior-to-posterior direction and PA represents images taken from a collimator in the posterior-to-anterior direction.

FIG. 5A-5D are images of transverse slices of a thorax region, abdominal region, coronal slice, and sagittal slice, respectively, in accordance with an example and taken with the system of FIG. 1.

FIGS. 6A and 6B illustrate depth dose profiles for a 10 mm collimator and a 5 mm collimator, respectively, showing plots of absolute dose rate versus depth, along the X-axis.

FIGS. 6C and 6D illustrate dose profiles for a 10 mm collimator and a 5 mm collimator, respectively, showing plots of relative dose versus distance from an isocenter along both the Y-axis and Z-axis.

FIG. 7 illustrates an example computer system for controlling the system of FIG. 1, in accordance with an example.

### DETAILED DESCRIPTION

The technology advances over the past decade have led to the rapid evolution of radiation therapy, from conformal, intensity modulation to stereotactic delivery of radiation treatment. Image guidance has dramatically improved the tumor targeting accuracy and precision, and thus has become an indispensable component in the modern radiation therapy practice. Preclinical or translational animal studies intended to investigate radiobiology mechanisms and develop new cancer treatment strategies are needed to be performed in the context of stereotactic irradiation by mimicking the treatment scenarios on patients and minimizing the artifacts caused by radiation damage to normal tissues.

However current small animal irradiation systems are either not accurate due to lack of image guidance, or too expensive for common research laboratories to afford. Furthermore, the image-guided, small-animal irradiators currently available usually have to be operated by dedicated and specially trained operators—their image-guided animal irradiation process is not very straightforward for typical animal researchers. In addition, some system has to compromise image guidance quality because of the complex design, resulting in non-optimal irradiation precision and accuracy.

With the present techniques, we have developed an integrated imaging and radiation research platform, that in an example implementation we refer to as an “image-guided small animal arc radiation treatment system” or “iSMAART”.

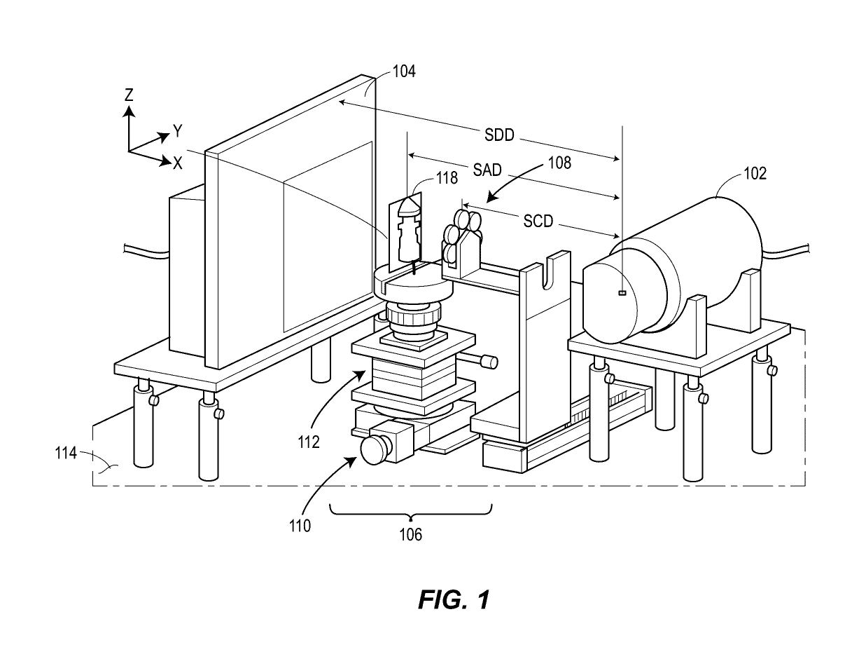

FIG. 1 illustrates an example small-animal radiation treatment system 100 that includes an x-ray tube 102, a flat panel detector 104, an animal stage 106, and a collimation subsystem 108. The system 100 is characterized, in at least some implementations, by the stationary nature of some components. In particular, unlike conventional systems, the system 100 maintains all imaging components and radiation components in a stationary configuration, i.e., in relation to an external testing bench or platform 114. Only a small animal mount is made to move during operation, in rotational direction and/or translational direction via the animal stage 106. By holding the x-ray tube 102, the imaging panel 104, and the collimation subsystem 108 stationary during operation more precise (cleaner) imaging can be achieved (in an imaging mode), as can more targeted radiation treatment (in a treatment mode).

The focus of the radiation from the x-ray 102 tube is switchable, in an example between 0.4 mm and 3 mm spot size. Any number of collimation diameters (and spot sizes) may be used, e.g., 0.4 mm, 0.5 mm, 0.6 mm, 0.7 mm, 0.8 mm, 0.9 mm, 1 mm, 2 mm, 3 mm, 5 mm, 7 mm, 8 mm, 10 mm, 15 mm, etc. In an imaging mode, the system 100 uses the small spot size focus, and corresponding collimator lens element. In a treatment mode, the system 100 uses the large spot size focus, and corresponding collimator lens element, for applying radiation to the small animal. In some examples, a collimator lens element is made to rotate to place the appropriate collimator into position in different mode operations. In some examples, the entire collimation subsystem 108 is moved out of the radiation field during imaging mode.

As noted, the x-ray tube 102 and image detector 104 are stationary, and the animal stage 106 is capable of fast rotation around z direction and 3-directional translation along x-y-z directions, using the animal stage 106 controlled by a computer system (not shown). The animal stage 106 includes a rotational subsystem 110 and a translational subsystem 112, which can be separate subsystems as described or can be combined into a single subsystem. As described, the subsystems 110 and 112 may be computer controllable for separate rotational movement and translational movement, respectively, to move a sample mounting stage 118 between treatment and imagine modes. As such, movement of the sample may be automated and performed in response to given changes in modes, or in response to misalignment of the sample from an isocenter over time, or in response to other events or triggers.

In the illustrated example, the translation subsystem 112 sits on top of the rotation subsystem 110, which is fixed on a benchtop 114, and provides a determined intersectional point between the central axis of radiation beams and the axis of animal rotation. This intersection point is defined as the system's isocenter for both CT imaging (or a planar radiograph) and stereotactic radiation delivery. In the illustrated example, the sample mount 118 is mounted on the translation subsystem 112 for holding the small animal in proper relation to this isocenter.

The collimation subsystem 108 is freestanding from the animal stage 106 and includes a filter wheel holding various sized collimators that can be remotely moved out of the radiation field during CT scan and into the radiation field to shape the beam aperture during irradiation. For example, the collimation subsystem 108 may be controlled by a motorized linear actuator also controlled by the computer system (not shown). In the illustrated example, six collimators have been inserted into the filter wheel and an appropriate collimator is selected depending on the actual target size, by the computer system. In another example, the filter wheel may be replaced by one or more motorized beam shaping collimators controlled by the computer system.

In operation, the system 100 is designed to control at least three distances: the source-to-detector distance (e.g., SDD=536 mm), source-to-axis distance (e.g., SAD=343 mm), and the source-to-collimator distance (e.g., SCD=250 mm), as shown in FIG. 1.

The system 100 is capable of fast volumetric imaging with a cone beam CT (CBCT) technique. The animal is positioned in the sample mount 118 (e.g., a specifically designed holder) and is rotated 360° for the acquisition of x-ray projections with the flat panel detector 104, while the x-ray tube 102 and detector panel 104 remain stationary.

The cone beam CT may be reconstructed with GPU accelerated FDK reconstruction algorithm (named after Feldkamp, Davis, and Kress (1984)) with less than 30 seconds. The total imaging time, including both projection acquisition and image reconstruction, was only about 1 minute, in an example implementation. We specifically imaged a prostate tumor. After the tumor was identified and localized from CT, the animal stage 106 (the translational stage 112 and the rotation stage 110) was shifted to align the tumor to the radiation isocenter. This shifting can be done manually, although in some examples the shifting is performed automatically, using a CT image analysis module coupled to or part of the computer system. Once the tumor has been identified, edge detection with contrast CT may be used, for example, to identify the tumor and a geometric analysis is performed to determine a geometric center or center line of the tumor, which is then used to align the tumor (and correspondingly the animal) to the radiation isocenter of the system 100. This may be performed as part of an imaging mode. While CT imaging and analysis is described in these examples, any suitable imaging modality may be used, including for example, two dimensional planar radiographs such as X-ray planar radiographs.

Imaging, alignment, image analysis are performed in the imaging mode. The system also includes a treatment mode in which a stereotactic radiation treatment can be delivered precisely and accurately to subject tissue by rotating the animal while radiation beams are provided at pinpointed tumor targets.

In an example implementation, a typical 2 Gy treatment can be finished with a beam in less than 1 minute. Table 1 shows the major parameters for CT imaging and stereotactic radiation, in an example implementation.

TABLE 1
Example System Parameters

| Parameters | CT imaging | Stereotactic radiation |
|---|---|---|
| Tube voltage (kVp) | 40-80 | 225 |
| Tube current (mA) | 1-6 | 13 |
| Focal spot size (mm) | 0.4 | 3 |
| Filtration | 0.1 mm Cu | 0.3 mm Cu/2 mm Al |
| Radiation dose (cGy) | 1-4 | 100-500 min⁻¹ |
| Time | ~1 minute | Depends on dose |

We have designed and performed rigorous experiments to validate the design and to assess the system capability of high quality CT imaging, image guidance, precision targeting, animal positioning, and stereotactic radiation.

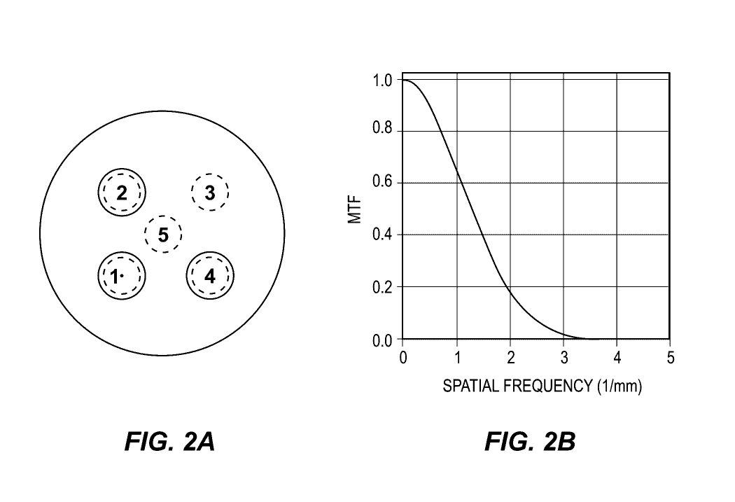

CT imaging. We designed and fabricated specific phantoms to calibrate our CT imaging subsystem and to assess image quality. According to our experiment results, the CT imaging system achieves high signal-to-noise ratio (SNR) and contrast-to-noise ratio (CNR), which are sufficient to differentiate major organ types. The high contrast spatial resolution was about 300 μm. FIGS. 2A and 2B illustrate the CT image quality. As FIG. 2A shows, the present techniques able to clearly differentiate four (4) heterogeneities embedded in an acrylic testing cylinder (air, polyethylene, delrin, and Teflon). The modulation transfer function (MTF) showing the spatial resolution is plotted in FIG. 2B.

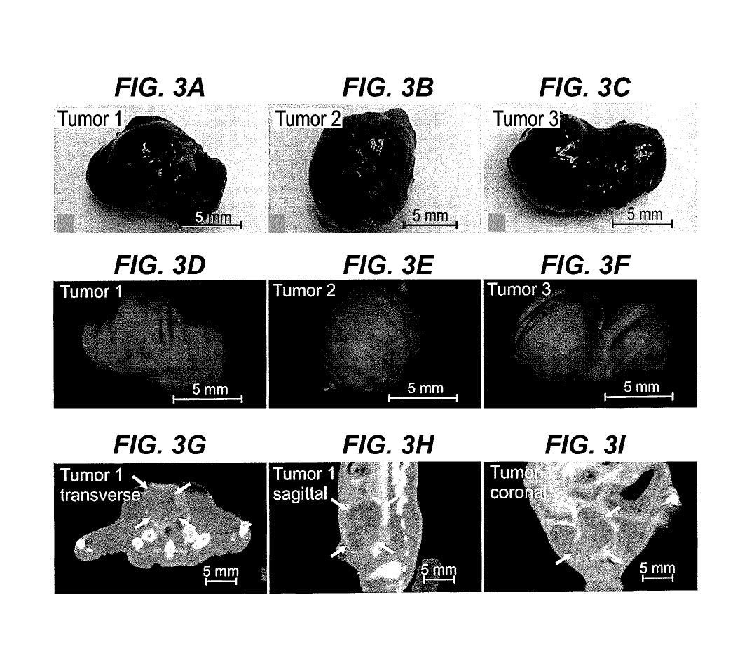

Image guidance. We imaged mice with a tumor to confirm CT's capability of differentiating tumors from healthy tissues. The tumor shapes from the 3D rendering of tumor contours in contrast CT are shown in FIGS. 3D-3F and match those of the actual tumors excised from sacrificed animals, as shown in FIGS. 3A-3C. As an example, the contrast enhancement pattern of the tumor #1 was shown in FIGS. 3G-3I with the arrows indicating the tumor edge enhanced by iodine based CT contrast agent. Moreover, image guidance can also be provided by imaging animals with two-dimension x-ray projection images.

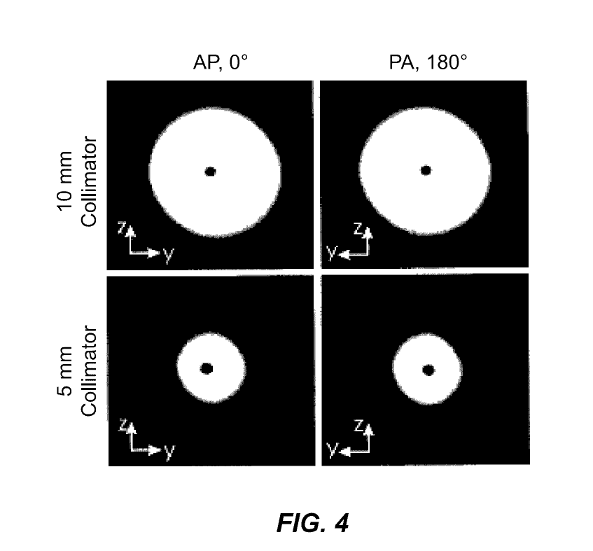

Precision targeting. We confirmed the precision of CT guided radiation targeting by imaging a steel BB with CT, shifting the BB to the radiation isocenter, and then targeting the BB with radiation beams. The overall precision in an example was less than 0.2 mm. FIG. 4 shows the exposure from two radiation beams targeting the BB from two opposite directions. AP represents images taken from a collimator in the anterior-to-posterior direction. PA represents images taken from a collimator in the posterior-to-anterior direction. The radiopaque BB is shown in the central black spot, while the white surrounding areas is from the radiation exposure of the collimated beam. The small deviation from the beam center from the BB center confirms the precision delivery of radiation beams to the assumed target within a less than 0.2 mm accuracy.

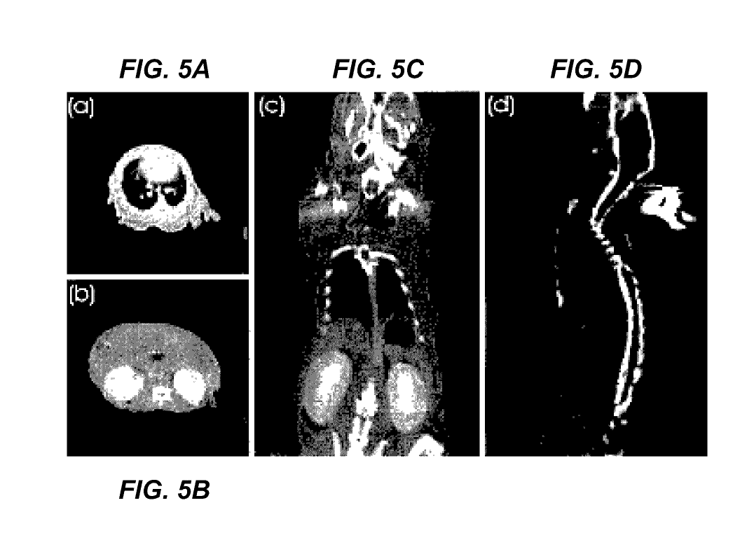

Animal positioning. Our system utilizes a unique animal geometry for both imaging and radiation. The animal can be hung up or tiled at any angle from 0°-90° with specially customized animal holders. We performed animal experiments and validated that the animal setup is stable during the entire image guided radiation process. As shown in FIGS. 5A-5D, the image blend between the first CT image (around 1 minute after setup) and last CT image (at 15 minutes after setup) indicates an almost perfect registration. That is, each of FIGS. 5A-5D is a composite of multiple images taken minutes apart and of a transverse slice of a thorax (FIG. 5A), a transverse slice of an abdomen (FIG. 5B), a coronal slice (FIG. 5C), and a sagittal slice (FIG. 5D), all of a mouse. The high definition of these composite images confirms the stability of our imaging and radiation geometry.

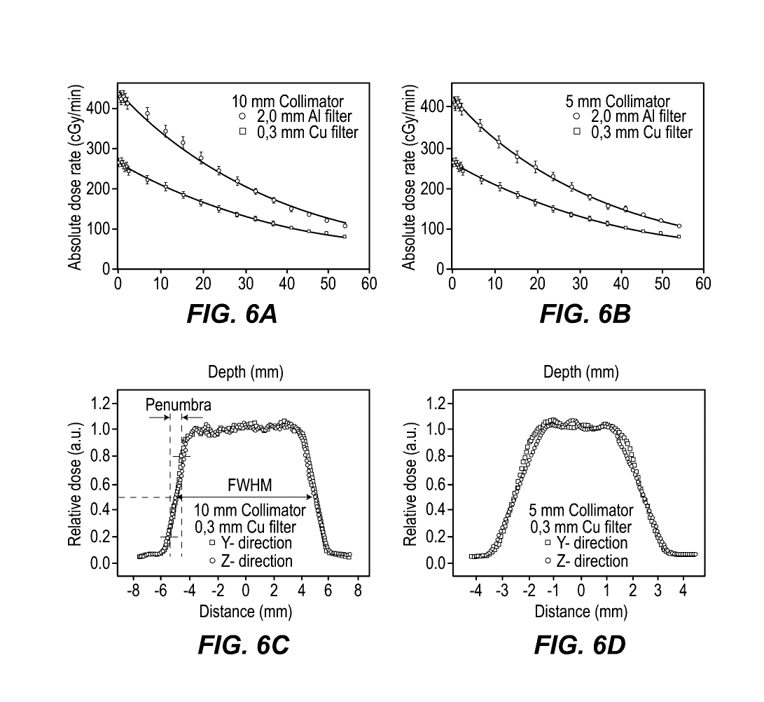

Stereotactic radiation. An advantage of the present techniques is the convenient and robust delivery of stereotactic radiation beams from arbitrary angles in a 360° volumetric arc during the treatment mode. The radiation source is stationary while the animal is rotated with the tumor/treating target positioned in the isocenter. Because the techniques do not use any heavy gantry as in other image guided irradiators, there is no gantry flex and the targeting process is very precise and robust. FIGS. 6A and 6B show the absolute dose rate at different depths for 5 mm (FIG. 6B) and 10 mm (FIG. 6A) collimators, for both Al and Cu filters in an example, during an example treatment mode. We also measured their dose profiles such as beam size and penumbra (FIG. 6C and FIG. 6D). FIG. 6C shows the relative dose amounts (a.u.) at different distances from the isocenter along the Y-axis and the Z-axis, defined in FIG. 1. FIG. 6C shows the dose profile for an example treatment using a 10 mm (diameter) collimator and a 0.3 mm Cu filter. The penumbra, along with the full width half max (FWHM), is as shown. FIG. 6D illustrates a dose profile but using a 5 mm (diameter) collimator.

The zero (“0”) distance for the plots in FIGS. 6C and 6D is at a given isocenter position, and may represent a point at a particular X-axis depth inside tissue to be treated.

The metallic filter is optional. Furthermore, when a filter is used, the thickness and material may be selected to control the dose fall-off, i.e., the penumbra, of the dose profile. Moreover, dose profile fall-off may be selected depending on the subject or tissue to be treated. For example, a thinner Cu filter (0.3 mm) may be chosen when treating a larger animal such as a rat or rabbit, while a thick 2 mm Al filter may be used for irradiating mice and other smaller subjects. In any event, based on the foregoing we can prescribe correct beam on time for a treating target located at any depth given a radiation dose. FIGS. 6C and 6D show the absolute depth dose rate for 5 mm and 10 mm collimators only.

The present techniques may use one single x-ray source for both volumetric imaging and radiation delivery. They do not use a rotating gantry for either CT imaging or radiation delivery. With this smart configuration, the unique designs offer significant advantages: 1) significant reduction in the cost of the device and operation; 2) simplified mechanical control and radiation shielding; 3) improved geometrical robustness in imaging and radiation delivery; 4) convenient delivery of stereotactic radiation beams; 5) easy to couple the other imaging modalities with micro-CT for multiple modality imaging and image guidance; and 6) it is a self-shielded mobile system and can be moved around and accommodated to any location inside one research facility.

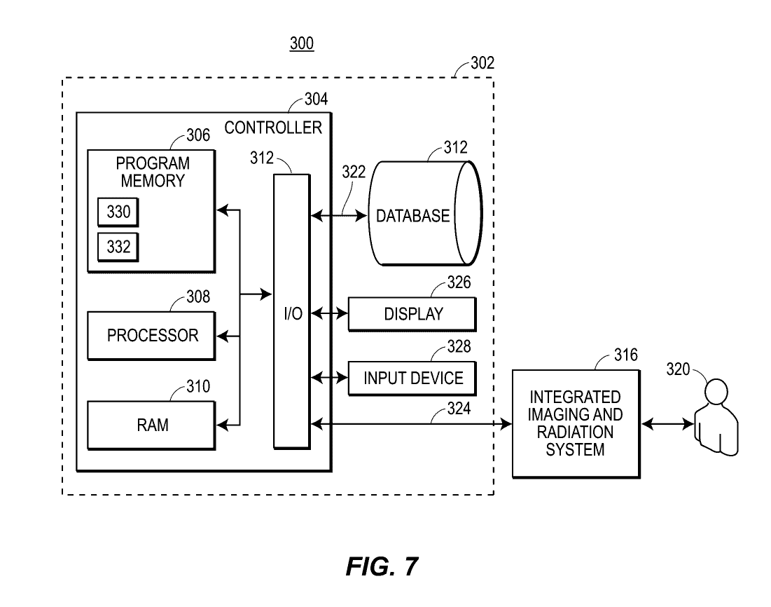

FIG. 7 illustrates an example computer system 300 that may be implemented to control the stages and subsystems of the system 100 in FIG. 1, in accordance with an example. A control device 302 is coupled to an integrated imaging and radiation system 316, like that of the system 100, is used in analyzing a subject 320. That subject 310 may be mounted to the integrated imaging and radiation system 316, as exemplified in FIG. 1. The control device 302 may have a controller 304 operatively connected to a database 314 via a link 322 connected to an input/output (I/O) circuit 312. It should be noted that, while not shown, additional databases may be linked to the controller 304 in a known manner. The controller 304 includes a program memory 306, the processor 308 (may be called a microcontroller or a microprocessor), a random-access memory (RAM) 310, and the input/output (I/O) circuit 312, all of which are interconnected via an address/data bus 320. It should be appreciated that although only one microprocessor 308 is shown, the controller 304 may include multiple microprocessors 308. Similarly, the memory of the controller 304 may include multiple RAMs 310 and multiple program memories 306. Although the I/O circuit 312 is shown as a single block, it should be appreciated that the I/O circuit 312 may include a number of different types of I/O circuits. The RAM(s) 310 and the program memories 306 may be implemented as semiconductor memories, magnetically readable memories, and/or optically readable memories, for example. A link 324 may operatively connect the controller 304 to the system 316 through the I/O circuit 312.

The program memory 306 and/or the RAM 310 may store various applications (i.e., machine readable instructions) for execution by the microprocessor 308. For example, an operating system 330 may generally control the operation of the control device 302 and provide a user interface to the control device 302 to implement the movement controls and/or imaging analyses described herein. The program memory 306 and/or the RAM 310 may also store a variety of subroutines 332 for accessing specific functions of the control device 302. By way of example, and without limitation, the subroutines 332 may include, among other things: a subroutine for controlling rotational movement of an animal stage, a subroutine for controlling translational movement of the animal stage, a subroutine for controlling isocenter alignment of a translational stage, a subroutine for controlling collimation subsystem movement and operation, a subroutine for controlling x-ray tube operation including radiation dosage amount, a subroutine for controlling image analysis, and other subroutines, for example, implementing software keyboard functionality, interfacing with other hardware in the control device 302, etc.

The program memory 306 and/or the RAM 310 may further store data related to the configuration and/or operation of the control device 302, and/or related to the operation of one or more subroutines 332. For example, image data may be data gathered by the device 316, data determined and/or calculated by the processor 308, etc. In addition to the controller 304, the control device 302 may include other hardware resources. The control device 302 may also include various types of input/output hardware such as a visual display 326 and input device(s) 328 (e.g., keypad, keyboard, etc.). In an embodiment, the display 326 is touch-sensitive, and may cooperate with a software keyboard routine as one of the software routines 332 to accept user input. It may be advantageous for the apparatus to communicate with broader medical analysis networks or medical treatment networks (not shown) through any of a number of known networking devices and techniques (e.g., through a commuter network such as a hospital or clinic intranet, the Internet, etc.). For example, the control device may be connected to a medical records database, hospital management processing system, health care professional terminals (e.g., doctor stations, nurse stations), patient monitoring systems, automated drug delivery systems. Accordingly, the disclosed embodiments may be used as part of an automated closed loop system or as part of a decision assist system.

As described, the controller device 302 may be configured to control the subject stage 106 for rational and translatory movement, whether achieved separately or simultaneously. That control may be achieved from instructions generated at the controller 304 from stored treatment and imaging protocol data. Or treatment and imaging protocol instruction may be pre-stored. In either case, the instructions may include data such as imaging exposure times, treatment exposure times, rotation position for imaging, rotation position for treatment, translation position for imaging, translation position for treatment, rotation speed, translation speed, and alignment data. Some data may be determined during setup, such as isocenter of the subject (e.g., “0” position in the YZ-plane).

During operation, the controller 304 may determine isocenter offset when the subject becomes misaligned. Offset may be determined from comparing collected image data, e.g., comparing current image data against an initial aligned subject's image data. To that end, image markers may be used for one or both of the Z-axis and the Y-axis. The markers may be axis lines on a support plate to which the subject is mounted. In other examples, markers are placed physically on the subject. In yet other examples, an initial image is taken of the subject and known filtering and feature recognition image processing algorithms are applied to identify features and positions. In subsequent images, these features are identified and positioned, whereafter this new position data is compared to the initial position data. In any of the examples, known imaging processing misalignment measurement techniques may be used to determine isocenter offset or feature offset and to determine corresponding rotational and translation adjustment needed to correct the offset.

The system 300 may be configured to control radiation treatment (dose application, collimator and spot size, isocenter position, subsurface dosage positions (X-axis), dosage angle of incidence, etc.) in the treatment mode. The system 300 may be configured to control movement of the subject from the treatment position to the imaging position, where the system 300 collects one or more images of the treated subject and can perform image processing and analysis, in some examples. This process can repeat through an entire treatment regimen, with the system 300 moving a target tissue between treatment and imaging positions has a treatment regimen is applied over time.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as examples and does not describe every possible embodiment, as describing every possible embodiment would be impractical, if not impossible. One could implement numerous alternate embodiments, using either current technology or technology developed after the filing date of this application.

What is claimed:

1. An x-ray radiation treatment and analysis system for use in vivo testing of a subject tissue, the system comprising:
a subject mounting stage having a rotational subsystem configured to rotate about a central axis and having a translational subsystem configured to move in translatory manner along at least two orthogonal directions;
an image plate configured to image computed tomography images or a planar radiograph collected of a tissue of the subject during an imaging mode of the system;
a collimating stage mounted to receive x-ray radiation from an x-ray source and configured to collimate the received x-ray radiation into a desired aperture size and direct the collimated x-ray radiation onto the tissue of the subject, where the collimating stage is adapted (i) to adjust between a first desired aperture size for use during an imaging mode and a second desired aperture size for use during a treatment mode, or (ii) to automatically move out of a path of the x-ray radiation during the imaging mode,
wherein the image plate and the collimating stage are maintained in a fixed, not moving position during the imaging mode and during the treatment mode, and wherein the subject mounting stage is controlled to move to place the subject in proper position for imaging during the image mode and for treatment during the treatment mode; and
a controller having at least one processor and at least one memory, the controller being coupled to control movement of the subject mounting stage in switching between operation of the system in the imaging mode and the treatment mode.

2. The system of claim 1, wherein collimating stage comprises a plurality of collimating lens elements mounted on a rotating wheel and positioned to move into and out of a radiation beam path and wherein collimating lens elements produce different collimating beam sizes.

3. The system of claim 1, wherein the rotational subsystem of the subject mounting stage is able to rotate the subject in 360 degrees of rotation.

4. The system of claim 3, wherein the translational subsystem of the subject mounting stage is able to perform translatory movement in three orthogonal directions.

5. The system of claim 4, wherein the controller is configured to control the subject mounting stage for simultaneous rotational and translatory movement of the subject.

6. The system of claim 5, wherein the controller is configured to determine an isocenter offset of the subject and to perform rotational and/or translatory movement of the subject until the subject is aligned on an isocenter of the subject mounting stage.

7. The system of claim 1, wherein the controller is configured to move the subject mounting stage during the treatment mode to deliver to the subject tissue treatment radiation dosages from different angles of incidence.

8. The system of claim 7, wherein the controller is configured to image the subject tissue, during the imaging mode, the effects of the delivered treatment radiation dosages from each of the different angles of incidence.
